(19) United States
(12) Patent Application Publication
(10) Pub. No.: US 2022/0008018 A1
(43) Pub. Date: Jan. 13, 2022

(54) SYSTEM AND METHOD FOR PREDICTING RISK OF ACUTE RENAL FAILURE FOLLOWING NON-CARDIAC SURGERY

(71) Applicant: SEOUL NATIONAL UNIVERSITY HOSPITAL, Seoul (KR)

(72) Inventors: Ha Jeong LEE, Seoul (KR); Se Hoon PARK, Seoul (KR); Yon Su KIM, Seoul (KR)

(21) Appl. No.: 17/294,657

(22) PCT Filed: Nov. 18, 2019
(86) PCT No.: PCT/KR2019/015727

§ 371 Date: May 17, 2021

Related U.S. Application Data
(60) Provisional application No. 62/768,221, filed on Nov. 16, 2018.

(30) Foreign Application Priority Data
Nov. 4, 2019 (KR) 10-2019-0139378

(51) Int. Cl. A61B 5/00; A61B 5/20; G16H 50/30; G16H 50/70; G16H 50/20

(52) U.S. Cl. CPC A61B 5/7275; A61B 5/201; G16H 70/20; G16H 50/70; G16H 50/20; G16H 50/30

(57) ABSTRACT

A system for predicting the risk of acute kidney injury after non-cardiac surgery according to an embodiment includes a variable selection unit, a classification reference point setting unit, and a prediction unit for the risk of acute kidney injury after non-cardiac surgery. The method includes: a first step of selecting variables, a second step of setting a classification reference point, and a third step of predicting the risk of acute kidney injury after non-cardiac surgery. The present invention is one that is simple and accurate, thereby attaining high applicability in clinical field.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2019/015727, filed Nov. 18, 2019, which claims priority to the benefit of US Patent Application No. 62/768,221 filed on Nov. 16, 2018 and Korean Patent Application No. 10-2019-0139378 filed in the Korean Intellectual Property Office on Nov. 4, 2019, the entire contents of which are incorporated herein by reference.

### BACKGROUND

### 1. Technical Field

The present invention relates to a system and method for predicting a risk of acute kidney injury after non-cardiac surgery.

### 2. Background Art

Acute kidney injury after surgery is a significant event associated with the death/dialysis/extended hospital stay of a patient after surgery, which occurs at a rate of 1 to 30% in patients who underwent surgery. It is known from previous reports that, when acute kidney damage occurs, a risk of short- or long-term death or a risk of dialysis due to renal failure is increased, and a hospital staying period and medical expenses during hospital staying are also significantly increased. There have been many reports of kidney damage after heart surgery in previous studies, but studies on kidney damage after non-cardiac surgery have been relatively rare. In particular, a prediction model for evaluating patients at a high risk of acute kidney injury after surgery and predicting the risk of occurrence has been little reported. Among them, a prediction model using a “clinical easy-to-use score system” that “has demonstrated its validity in an independent patient group” has not yet been reported.

### SUMMARY

It is an object of the present invention to provide a system and method for predicting a risk of acute kidney injury after non-cardiac surgery using non-cardiac clinical data from non-cardiac patients before non-cardiac surgery.

To achieve the above objects, the following technical solutions are adopted in the present invention.

1. A system for predicting a risk of acute kidney injury after non-cardiac surgery, the system including: a variable selection unit configured to select factors associated with an occurrence of acute kidney injury after non-cardiac surgery, which include clinical data before non-cardiac surgery of patients subjected to non-cardiac surgery as variables, and preset an index set for each variable;

a classification reference point (“classification cutoff value”) setting unit configured to calculate sensitivity and specificity for a sum of combinations of indexes for each index set, and set cutoff values according to the sensitivity and specificity; and

a prediction unit for the risk of acute kidney injury after non-cardiac surgery, which is configured to calculate a sum of indexes determined according to the index set preset for each variable selected by the variable selection unit in regard to a patient subjected to non-cardiac surgery in need of a prediction, and then, predict the risk of acute kidney injury after non-cardiac surgery of the non-cardiac surgery patient in need of the prediction, on the basis of cutoff values set in the classification cutoff value setting unit,

wherein the factors associated with the occurrence of acute kidney injury after non-cardiac surgery include at least one selected from the group consisting of age, estimated glomerular filtration rate (eGFR), dipstick albuminuria, sex, expected surgical duration, emergency operation, diabetes mellitus, use of renin-aldosterone-angiotensin-system blocker (use of RAAS blocker), hypoalbuminemia, anemia and hyponatremia.

2. The system according to the above 1, wherein the index set is preset according to a selection category for each variable except for the expected surgical duration, at least one index selected from 0, 3, 4, 6 to 9, 13, 15 and 22 is preset for each selection category, and a sum of the indexes for each selection category of the variable is 0 to 81.

3. The system according to the above 1, wherein the index preset for the expected surgical duration among the variables is set to 5 times the expected surgical duration (hours).

4. The system according to the above 1, wherein, among the variables, the age is divided into selection categories of less than 40 years old, 40 or more and less than 60 years old, 60 or more and less than 80 years old, and not less than 80 years old, and

wherein the estimated glomerular filtration rate (eGFR) is divided into selection categories of 60 mL/min/1.73 m² or more, 45 mL/min/1.73 m² or more and less than 60 mL/min/1.73 m², 30 mL/min/1.73 m² or more and less than 45 mL/min/1.73 m², 15 mL/min/1.73 m² or more and less than 30 mL/min/1.73 m².

5. The system according to the above 1, wherein the factors associated with the occurrence of acute kidney injury after non-cardiac surgery include age, estimated glomerular filtration rate (eGFR), dipstick albuminuria, sex, expected surgical duration, emergency operation, diabetes mellitus, use of renin-aldosterone-angiotensin-system blocker, hypoalbuminemia, anemia and hyponatremia.

6. The system according to the above 1, wherein the prediction of the risk of acute kidney injury after non-cardiac surgery is classified into total four (4) grades including A, B, C and D, based on a sum of indexes determined according to the index set preset for each variable selected in the variable selection unit in regard to the non-cardiac surgery patient in need of the prediction.

7. The system according to the above 6, wherein the grade A is classified when the sum of the indexes is less than 20, the grade B is classified when the sum of the indexes is 20 or more and less than 40, the grade C is classified when the sum of the indexes is 40 or more and less than 60, and the grade D is classified when the sum of the indexes is 60 or more.

8. The system according to the above 7, wherein the grade A involves less than 2% probability of both acute kidney injury and severe acute kidney injury after non-cardiac surgery of patients subjected to non-cardiac surgery (“non-cardiac surgery patients”) in need of the prediction;

the grade B involves 2% or more probability of acute kidney injury and less than 2% probability of serious acute kidney injury after non-cardiac surgery of the non-cardiac surgery patients in need of the prediction;

the grade C involves 10% or more probability of acute kidney injury and 2% or more probability of serious acute kidney injury after non-cardiac surgery of the non-cardiac surgery patients in need of the prediction; and

the grade D involves 20% or more probability of acute kidney injury and 10% or more probability of serious acute kidney injury after non-cardiac surgery of the non-cardiac surgery patients in need of the prediction.

9. A method for predicting a risk of acute kidney injury after non-cardiac surgery, the method including: a first step of selecting factors associated with an occurrence of acute kidney injury after non-cardiac surgery, which consist of clinical data before non-cardiac surgery of patients subjected to non-cardiac surgery as variables, and then, presetting an index set for each variable;

a second step of calculating sensitivity and specificity to a sum of combinations of indexes for each index set, and setting cutoff values for classification according to the sensitivity and specificity; and

a third step of calculating a sum of indexes determined according to the index set preset for each variable selected in the first step in regard to the non-cardiac surgery patient in need of a prediction, and then, predicting a risk of acute kidney injury after non-cardiac surgery of the non-cardiac surgery patient on the basis of the cutoff values set in the second step,

wherein the factors associated with the occurrence of acute kidney injury after non-cardiac surgery include at least one selected from the group consisting of age, estimated glomerular filtration rate (eGFR), dipstick albuminuria, sex, expected surgical duration, emergency operation, diabetes mellitus, use of renin-aldosterone-angiotensin-system blocker (use of RAAS blocker), hypoalbuminemia, anemia and hyponatremia.

10. The method according to the above 9, wherein the index set is preset according to a selection category for each variable except for the expected surgical duration, at least one index selected from 0, 3, 4, 6 to 9, 13, 15 and 22 is preset for each selection category, and a sum of the indexes for each selection category of the variable is 0 to 81.

11. The method according to the above 9, wherein the index preset for the expected surgical duration among the variables is set to 5 times the expected surgical duration (hours).

12. The method according to the above 9, wherein, among the variables, the age is divided into selection categories of less than 40 years old, 40 or more and less than 60 years old, 60 or more and less than 80 years old, and not less than 80 years old, and wherein the estimated glomerular filtration rate (eGFR) is divided into selection categories of 60 mL/min/1.73 m² or more, 45 mL/min/1.73 m² or more and less than 60 mL/min/1.73 m², 30 mL/min/1.73 m² or more and less than 45 mL/min/1.73 m², 15 mL/min/1.73 m² or more and less than 30 mL/min/1.73 m².

13. The method according to the above 9, wherein the factors associated with the occurrence of acute kidney injury after non-cardiac surgery include age, estimated glomerular filtration rate (eGFR), dipstick albuminuria, sex, expected surgical duration, emergency operation, diabetes mellitus, use of renin-aldosterone-angiotensin-system blocker (use of RAAS blocker), hypoalbuminemia, anemia and hyponatremia.

14. The method according to the above 9, wherein the prediction of the risk of acute kidney injury after non-cardiac surgery is classified into total four (4) grades including A, B, C and D, based on a sum of indexes determined according to the index set preset for each variable selected in the variable selection unit in regard to the non-cardiac surgery patient in need of the prediction.

15. The method according to the above 14, wherein the grade A is classified when the sum of the indexes is less than 20, the grade B is classified when the sum of the indexes is 20 or more and less than 40, the grade C is classified when the sum of the indexes is 40 or more and less than 60, and the grade D is classified when the sum of the indexes is 60 or more.

16. The method according to the above 15, wherein the grade A involves less than 2% probability of both acute kidney injury and severe acute kidney injury after non-cardiac surgery of patients subjected to non-cardiac surgery (“non-cardiac surgery patients”) in need of the prediction;

the grade B involves 2% or more probability of acute kidney injury and less than 2% probability of serious acute kidney injury after non-cardiac surgery of the non-cardiac surgery patients in need of the prediction;

the grade C involves 10% or more probability of acute kidney injury and 2% or more probability of serious acute kidney injury after non-cardiac surgery of the non-cardiac surgery patients in need of the prediction; and

the grade D involves 20% or more probability of acute kidney injury and 10% or more probability of serious acute kidney injury after non-cardiac surgery of the non-cardiac surgery patients in need of the prediction.

According to the present invention, it is possible to predict acute kidney injury or relevant prognoses of a patient before actual surgery is executed with measurable/predictable indicators before surgery. Further, according to the present invention, it is possible to not only reduce the risk of short-term/long-term death, the risk of dialysis, etc., but also use an accurate, simple and easy scoring system, thereby attaining high applicability in clinical field.

### BRIEF DESCRIPTION OF THE DRAWINGS

FIG. 1 is a diagram summarizing an embodiment of the present invention.

FIG. 2 is a diagram illustrating a variable selection process implemented in discovery cohort.

FIG. 3 is a diagram illustrating compensation chart and a receiver-operating characteristic curve.

FIG. 4 is a diagram illustrating sensitivity and specificity to SPARK index.

FIG. 5 is a diagram illustrating SPARK risk classification and incidence of low-stage AKI and critical AKI.

FIG. 6 is a diagram illustrating a pre-operative PO-AKI risk assessment strategy based on the proposed SPAK classification.

FIGS. 7A and 7B are diagrams illustrating a calibration chart and a receiver-operating characteristic curve in each surgical department.

### DETAILED DESCRIPTION

Hereinafter, the present invention will be described in detail.

The present invention provides a system for predicting a risk of acute kidney injury after non-cardiac surgery.

The system of the present invention includes a variable selection unit configured to select factors associated with an occurrence of acute kidney injury after non-cardiac surgery, which include clinical data before non-cardiac surgery of patients subjected to non-cardiac surgery (“non-cardiac surgery patients”) as variables, and preset an index set for each variable.

The non-cardiac surgery means any surgery other than cardiac surgery.

The non-cardiac surgery preclinical data of the non-cardiac surgery patients may be acquired through at least one route selected from an electronic medical record (EMR) of a hospital, an interview with a patient or guardian, an inpatient nursing information record, a nursing activity report and an inpatient record, but it is not limited thereto.

The factors associated with the occurrence of acute kidney injury after non-cardiac surgery may be collected from a discovery cohort and a validation cohort, but it is not limited thereto.

The variable selection unit may consider statistical assumptions and multivariable analysis for a proportional odds regression technique and select factors with a large model coefficient as variables, but it is not limited thereto.

Further, the variable selection unit may construct a multivariable model in regard to ordinal variables composed of negative prognoses related to the acute kidney injury using the proportional odds regression technique with the selected variables and, at the same time, may preset an index set for each variable so that a sum of the indexes preset in each variable reflects the risk by converting model coefficients into an integer, but it is not limited thereto.

The factors associated with the occurrence of acute kidney injury after non-cardiac surgery may include at least one selected from the group consisting of age, estimated glomerular filtration rate (eGFR), dipstick albuminuria, sex, expected surgical duration, emergency operation, diabetes mellitus, use of renin-aldosterone-angiotensin-system blocker (use of RAAS blocker), hypoalbuminemia, anemia and hyponatremia, preferably, all of the above factors, but it is not limited thereto.

According to an embodiment of the present invention, an index set may be preset for each variable and for each index except for the expected surgical duration. The index set may be preset to (0, X), wherein X is any one index selected from 3, 4, 6 to 9, 13 15 and 22. For example, a patient may have one index in the index set according to definition of the variable. For example, when the index set of the sex variable is (0, 8), the index may be 10 when the patient is a male while being 0 when the patient is a female.

According to an embodiment of the present invention, each variable may vary in a size of the index set by the number of selection categories. For example, if a predetermined variable has three selection categories, the index set defined for the predetermined variable may be (0, Y, Z). At this time, Y and Z are values corresponding to any one of the above X, and Y does not have the same value as Z.

Specifically, the age may be divided into selection categories of less than 40 years old, 40 or more and less than 60 years old, 60 or more and less than 80 years old, and not less than 80 years old.

Further, the estimated glomerular filtration rate (eGFR) may be divided into selection categories of 60 mL/min/1.73 m² or more, 45 mL/min/1.73 m² or more and less than 60 mL/min/1.73 m², 30 mL/min/1.73 m² or more and less than 45 mL/min/1.73 m², 15 mL/min/1.73 m² or more and less than 30 mL/min/1.73 m².

More particularly, the age may be set to an index set of: 0 when the age is less than 40 years old; 6 when the age is 40 or more and less than 60 years old; 9 when the age is 60 or more and less than 80 years old; and 13 when the age is not less than 80 years old. Likewise, the estimated glomerular filtration rate (eGFR) may be set to an index set of: 0 when it is 60 mL/min/1.73 m² or more; 8 when it is 45 mL/min/1.73 m² or more and less than 60 mL/min/1.73 m²; 15 when it is 30 mL/min/1.73 m² or more and less than 45 mL/min/1.73 m²; and 22 when it is 15 mL/min/1.73 m² or more and less than 30 mL/min/1.73 m². Further, an index set of 6 and 0 may be preset if Dipstick albuminuria is present or not, respectively. Further, an index set of 8 and 0 may be preset if the sex is male or female, respectively. Further, an index set of 7 and 0 may be preset if there is an emergency operation or not, respectively. Further, an index set of 4 and 0 may be preset if diabetes mellitus is present or not, respectively. Furthermore, an index set of 6 and 0 may be preset if a renin-aldosterone-angiotensin-system blocker (RAAS blocker) is used or not, respectively. Furthermore, an index set of 8 and 0 may be preset if hypoalbuminemia is present or not, respectively. Furthermore, an index set of 4 and 0 may be preset if anemia is present or not, respectively. Furthermore, an index set of 3 and 0 may be preset if hyponatremia is present or not, respectively. Accordingly, a sum of indexes for each variable excluding the expected surgical duration may be calculated from the minimum of 0 to the maximum of 81.

Further, the unit of the expected surgical duration among the variables may be time (hour), and the expected surgical duration may be defined to an index of 5 times the corresponding period. For example, if the expected surgical duration is 3 hours, the index of this variable is 15.

Further, the system for predicting a risk of acute kidney injury after non-cardiac surgery according to the present invention includes a classification reference point (“classification cutoff value”) setting unit configured to calculate sensitivity and specificity for a sum of combinations of indexes for each index set, and set cutoff values according to the calculated sensitivity and specificity.

The classification cutoff value setting unit may set the classification cutoff value by calculating the sensitivity and specificity for the sum of combinations of indexes in each index set using a receiver-operating characteristic curve (ROC curve).

The index set means a set of indexes preset according to a size of selection category for each variable, and the combination of indexes for each index set means a combination of indexes according to the selection category of each corresponding variable except for the expected surgical duration acquired from clinical data of a non-cardiac surgery patient, wherein a sum of combinations of indexes for each index set excluding the expected surgical duration may be the minimum of 0 to the maximum of 81.

With regard to the sensitivity and specificity of each index combination sum, the sensitivity and specificity of a variable may be calculated from an area under the curve (“AUC”) value of each of the discovery cohort and the validation cohort through analysis of the receiver-operating characteristic curve (“ROC” curve).

Meanwhile, with regard to setting the classification cutoff value, ROC curve analysis may be conducted to obtain a prediction probability using an estimated regression coefficient value, which in turn calculates a sum of the sensitivity and specificity, thereby setting the classification cutoff value.

More specifically, a cutoff value may be set using a Youden index in ROC curve.

Further, the system for predicting the risk of acute kidney injury after non-cardiac surgery according to the present invention includes a prediction unit for the risk of acute kidney injury after non-cardiac surgery, which is configured to calculate a sum of indexes determined according to the index set preset for each variable selected by the variable selection unit in regard to a patient subjected to non-cardiac surgery (“the non-cardiac surgery patient”) in need of a prediction, and then, predict the risk of acute kidney injury after non-cardiac surgery of the non-cardiac surgery patient in need of the prediction, on the basis of cutoff values set in the classification cutoff value setting unit.

According to an embodiment of the present invention, the prediction of the risk of acute kidney injury after non-cardiac surgery may be classified into total four (4) grades including A, B, C and D based on the sum of the indexes determined according to an index set preset for each variable selected in the variable selection unit in regard to the non-cardiac surgery patients in need of the prediction.

The grade A may be classified when the sum of the indexes is less than 20, the grade B may be classified when the sum of the indexes is 20 or more and less than 40, the grade C may be classified when the sum of the indexes is 40 or more and less than 60, and the grade D may be classified when the sum of the indexes is 60 or more.

Further, according to an embodiment of the present invention, the grade A may involve less than 2% probability of both acute kidney injury and severe acute kidney injury after non-cardiac surgery of the non-cardiac surgery patients in need of the prediction; the grade B may involve 2% or more probability of acute kidney injury and less than 2% probability of serious acute kidney injury after non-cardiac surgery of the non-cardiac surgery patients in need of the prediction; the grade C may involve 10% or more probability of acute kidney injury and 2% or more probability of serious acute kidney injury after non-cardiac surgery of the non-cardiac surgery patients in need of the prediction; and the grade D may involve 20% or more probability of acute kidney injury and 10% or more probability of serious acute kidney injury after non-cardiac surgery of the non-cardiac surgery patients in need of the prediction.

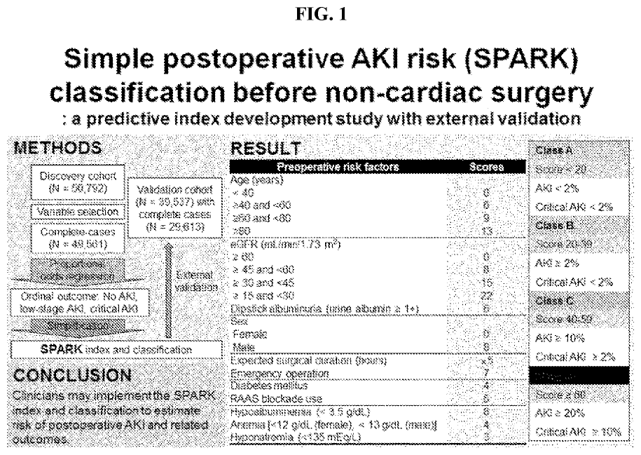

Further, the present invention provides a method for predicting a risk of acute kidney injury after non-cardiac surgery (see FIG. 1).

The method includes: a first step of selecting factors associated with an occurrence of acute kidney injury after non-cardiac surgery, which consist of non-cardiac surgery preclinical data of patients subjected to non-cardiac surgery as variables, and then, presetting an index set for each variable;

a second step of calculating sensitivity and specificity to a sum of combinations of indexes for each index set, and setting cutoff values for classification according to the sensitivity and specificity; and

a third step of calculating a sum of indexes determined according to the index set preset for each variable selected in the first step in regard to the non-cardiac surgery patient in need of a prediction, and then, predicting a risk of acute kidney injury after non-cardiac surgery of the non-cardiac surgery patient on the basis of the cutoff values set in the second step.

In this regard, the factors associated with the occurrence of acute kidney injury after non-cardiac surgery may include at least one selected from the group consisting of age, estimated glomerular filtration rate (eGFR), dipstick albuminuria, sex, expected surgical duration, emergency operation, diabetes mellitus, use of renin-aldosterone-angiotensin-system blocker (use of RAAS blocker), hypoalbuminemia, anemia and hyponatremia, and preferably all of the above factors, but it is not limited thereto.

According to an embodiment of the present invention, the index set may be preset according to a selection category for each variable except for the expected surgical duration, at least one index selected from 0, 3, 4, 6 to 9, 13, 15 and 22 may be preset for each selection category, and a sum of the indexes for each selection category of the variable may be 0 to 81.

Further, according to an embodiment of the present invention, the index preset for the expected surgical duration among the variables may be set to 5 times the expected surgical duration (hours).

Further, among the variables, the age may be divided into selection categories of less than 40 years old, 40 or more and less than 60 years old, 60 or more and less than 80 years old, and not less than 80 years old, and the estimated glomerular filtration rate (eGFR) may be divided into selection categories of 60 mL/min/1.73 m² or more, 45 mL/min/1.73 m² or more and less than 60 mL/min/1.73 m², 30 mL/min/1.73 m² or more and less than 45 mL/min/1.73 m², 15 mL/min/1.73 m² or more and less than 30 mL/min/1.73 m².

Further, according to an embodiment of the present invention, the prediction of the risk of acute kidney injury after non-cardiac surgery may be classified into total four (4) grades including A, B, C and D, based on a sum of indexes determined according to the index set preset for each variable selected in the variable selection unit in regard to the non-cardiac surgery patient in need of the prediction.

The grade A may be classified when the sum of the indexes is less than 20, the grade B may be classified when the sum of the indexes is 20 or more and less than 40, the grade C may be classified when the sum of the indexes is 40 or more and less than 60, and the grade D may be classified when the sum of the indexes is 60 or more.

According to an embodiment of the present invention, the grade A may involve less than 2% probability of both acute kidney injury and severe acute kidney injury after non-cardiac surgery of the non-cardiac surgery patients in need of the prediction; the grade B may involve 2% or more probability of acute kidney injury and less than 2% probability of serious acute kidney injury after non-cardiac surgery of the non-cardiac surgery patients in need of the prediction; the grade C may involve 10% or more probability of acute kidney injury and 2% or more probability of serious acute kidney injury after non-cardiac surgery of the non-cardiac surgery patients in need of the prediction; and the grade D may involve 20% or more probability of acute kidney injury and 10% or more probability of serious acute kidney injury after non-cardiac surgery of the non-cardiac surgery patients in need of the prediction.

The first, second, and third steps of the method described above, respectively, are substantially the same as performed by the variable selection unit, the classification cutoff value setting unit and the risk predicting unit for acute kidney injury after non-cardiac surgery included in the system of the present invention described above, and therefore, will not be described in detail below.

Hereinafter, examples will be described in detail to specifically describe the present invention.

### Examples

1. Experimental Method

(1) Hospital where the Experiment of the Present Invention was Implemented and Invention Design

The present invention provides a retrospective observation cohort experiment conducted at a government-designated tertiary care hospital in Korea. This discovery cohort includes adult patients over 18 ages old who have received surgery at Seoul National University Hospital between 2004 and 2013, and the validation cohort includes adults who have received surgery at Seoul National University Bundang Hospital between 2006 and 2015. Both hospitals have more than 1000 beds and top clinicians belonging to Seoul National University School of Medicine. However, the two hospitals are located in different administrative districts in Korea and do not share patient pools or major medical staffs.

First surgical cases are included during the experimental period in the following five surgical departments: General Surgery, Orthopedic Surgery, Gynecology, Neurosurgery and Urology. Exclusion criteria were as follows: 1) cardiac surgery, 2) surgery of a deceased patient (e.g. transplantation of a deceased donor), 3) patients with nephrectomy or kidney transplantation, 4) small surgical procedures defined with the surgical duration of less than 1 hour, 5) patients with pre-operative renal dysfunction which is defined by: history of kidney replacement therapy; pre-operative serum creatinine (sCr) level of 4 mg/dL or higher; estimated glomerular filtration rate (eGFR) of 15 mL/min/1.73 m² or an increase in baseline of sCr by 0.3 mg/dL or more or 1.5 times or more from the minimum value 2 weeks prior to surgery, and 6) patients without a baseline or subsequent sCr value to identify post-operative acute kidney injury (PO-AKI) troubles.

(2) Data Collection and Variables for Model Construction

Information that could be collected or planned before surgery was included because pre-operative risk classification is the object of the present invention. Most continuous variables were categorized by the ranges commonly used for practical application of the present invention. Detailed information on the collected variables is as follows

The following demographic data were collected from the discovery cohort and the validation cohort: age, sex and baseline body mass index (BMI) at the time of acquisition. Among them, age was categorized as <40, ≥40 and <60, ≥60 and <80 or ≥80 years old, and intervals were determined to limit the number of classifications for brevity. BMI was categorized into low weight (<18.5 kg/m²), normal range (≥18.5 and <25 kg/m²), and obesity (≥25 kg/m²). Co-morbidities of heart disease were collected, which include a history of heart failure, coronary artery disease (e.g., angina or myocardial infarction), hypertension and diabetes. The history of co-morbidities was mostly confirmed by reviewing the records of anesthesiologists, dosing of medications and diagnostic codes. In order to make out an anesthesia schedule and to reserve an operating room, data were collected in regard to the actual surgical duration (hours) and expected surgical duration (hours) entered by the physician who attended the collection prior to performing the surgery. The expected surgical duration only was included in the model because it is an obtainable variable before surgery. Anesthesia type (normal or non-normal) and whether the surgery was conducted as scheduled or as an emergency surgery were collected. Pre-operative systolic and diastolic blood pressures were recorded. Among the well-known PO-AKI related drugs, pre-operative use of renin-aldosterone-angiotensin-system blockers was included in the variables of the present invention. Diuretics, nonsteroidal anti-inflammatory drugs or nephrotoxic antibiotics were used frequently after surgery to control dose overload, pain or infection, therefore, were not collected. The collected laboratory values were results of the final examination within 3 months prior to surgery. Baseline eGFR was calculated based on sCr levels using CKD-EPI equation and then stratified into 4 classes (reference range=60 mL/min/1.73 m² or more; CKD 3A=45 mL/min/1.73 m² or more and less than 60 mL/min/1.73 m²; CKD 3B=30 mL/min/1.73 m² or more and less than 45 mL/min/1.73 m²; or CKD 4=15 mL/min/1.73 m² or more and less than 30 mL/min/1.73 m²). The presence of baseline proteinuria as another kidney function variable was confirmed by a simple dipstick test. Leukocyte count abnormality was classified into leukopenia (<4,000/μL) and leukocytosis (≥10,000/μL). Anemia was defined as a hemoglobin level of <12 g/dL for female and <13 g/dL for male. A serum albumin level below 3.5 g/dL is a definition for hypoalbuminemia. Reference electrolyte imbalance including hyponatremia (sodium in the blood <135 mEq/L), hypernatremia (sodium in the blood >145 mEq/L), hypokalemia (potassium in the blood <3.5 mEq/L) and hyperkalemia (potassium in the blood >5.5 mEq/L) was recorded.

(3) Experiment Results

PO-AKI was defined based on sCr-criterion of “Kidney Disease: Improving Global Outcomes guidelines” using peak values of sCr within 2 weeks after surgery. The term “PO-AKI” as used herein includes all AKIs regardless of AKI severity. In order to address the severity and patient-oriented outcomes of PO-AKI, the inventors defined results representing a sequential order to construct a prediction model that includes three outcome classifications as follows: “No AKI”, “Low-stage AKI” and “Critical AKI”. Among PO-AKI patients, critical AKI was defined by appearance of two or more AKI stages, which in turn, appears AKI causing death after AKI and dialysis within 90 days. When some patients began kidney transplant treatment outside the experimental hospital or died, the national death database from the Korean National Statistical Office and the national dialysis record maintained by the Korean Kidney Society were reviewed, followed by confirmation of the results. Other patients who developed stage 1 PO-AKI but did not have critical AKI were included in the “low-stage AKI” classification of the sequential results.

(4) Variable Selection

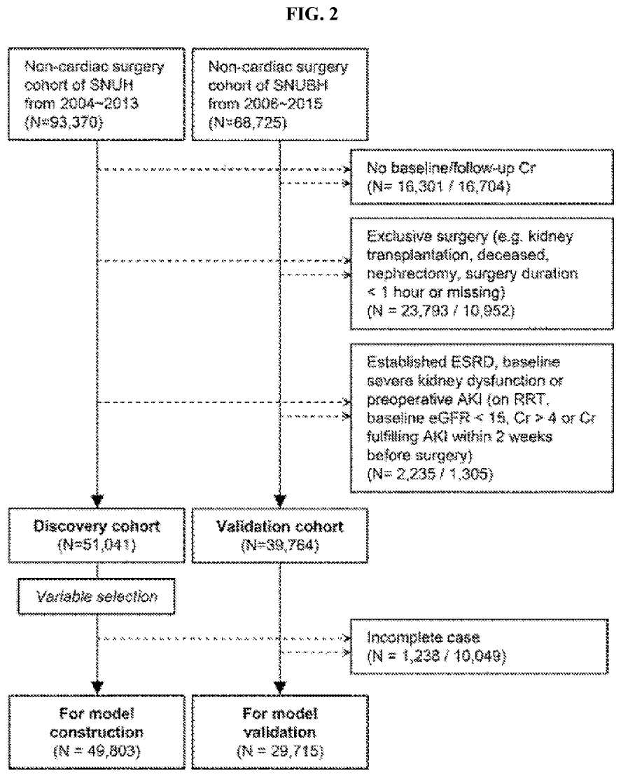

First, a variable selection process was conducted in the discovery cohort (FIG. 2). In order to identify variables that violated parallel regression estimation, a single-variant cumulative logistic regression analysis including binary results defined by different reference values in the sequential results was implemented. The parallel regression estimation was checked by examining a direction and a size of model coefficients rather than a statistical test which is typically semi-conservative in large data sets. Next, the sequential results were fitted to a model of multivariant proportional odds, and only variables appeared to have statistically significant relationships with independent and sequential results were left over. Finally, the number of variables included in the model according to absolute sizes of the model coefficients was reduced while excluding variables with relatively lower effects. After the variable selection, patients without missing values in the selected variables were subjected to further processes.

(5) Simple Postoperative AKI risK (SPARK) Index and Classification Thereof

Additional simplification was performed to construct a simple postoperative Aki RisK (SPARK) index after additional simple surgery. After confirming the calibration of the simple model, the coefficients were multiplied to set 100 as the maximum sum of model coefficients in the discovery cohort. Further, each coefficient was rounded to an integer to generate the SPARK index. Finally, in order to create a general classification that can be easily interpreted in practice, cutoff values to define four (4) classes were confirmed in the discovery cohort. The cutoff value for A/B grades was defined to suggest a threshold for PO-AKI screening with a high sensitivity (90%), while a threshold for B/C grades was PO-AKI was proposed to be a value with a high specificity (90%). Lastly, for patients with a SPARK index higher than the cutoff value for B/C grades, a cutoff value for C/D grades was further determined while a threshold with high specificity for critical AKI (90%) was selected to define the D grade. Considering practical problems, the threshold was rounded to the nearest value to 10.

(6) Sensitivity Analysis and Other Statistical Analysis

Other statistical analysis methods including sensitivity analysis were as follows.

Classification variables were represented as frequency (percentage), and continuous variables were expressed as the median (quartile range). Chi-squared test and Mann-Whitney U test were implemented to compare the basic characteristics of the discovery cohort and the validation cohort. Model calibration was first visually checked with a calibration plot of probability for both the estimated low-stage AKI and critical AKI. Since the present invention involved a large number of patients (more than 25000), applying Hosmer-Lemeshow test was basically not recommended. Therefore, P value of the Hosmer-Lemeshow test was calculated from thousands of random sub-samples with a fixed sub-sample size (n=1000). Discrimination of the model was confirmed by c-stat. The predictive ability of the SPARK index was subjected to final test with regard to PO-AKI and critical AKI results along with analysis of the receiver-operating characteristics curve (ROC curve). As a result, the area under the curve (AUC) value of 0.7 or higher was considered to be acceptable. In order to determine whether there was a significant bias due to the exclusion criteria, sensitivity analysis was performed. With regard to the analysis, a discriminative ability for the final SPARK index related to the sequential results was calculated along with: 1) the replaced data set using additional nonlinear transformations and substitutions; 2) input of the actual surgical duration instead of the expected one, 3) data sets divided according to three ages (2004 to 2007, 2008 to 2011, and 2012 to 2015) after merging of this experiment and the verification cohort; and 4) data sets remaining after additionally excluding the patients with sCr increased by 0.3 mg/dL or more or 1.5 times or higher than the lowest value within 3 months postoperatively after surgery, regardless of an interval between surgeries in order to strictly control possible inclusions of patients having undefined AKI before surgery. Further, the performance of SPARK index and classification thereof was examined in each surgical department after combining the discovery cohort and the validation cohort. All analyses were performed in perfect cases with no missing values except for sensitivity analysis with attributed data sets. Statistical analyses were performed with R (version 3.4.3, the R foundation), and two-sided P values <0.05 were considered statistically significant.

2. Experimental Results

(1) Characteristics of the Cohort Tested

A total of 162,095 patients were included in the screening in the present experiment, which was the sum of 93,370 and 68,725 surgical cases tested in SNUH (Seoul National University Hospital) and SNUBH (Seoul National University Bundang Hospital), respectively (FIG. 2). After the exclusion criteria were applied, 51,041 and 39,764 patients were screened for model construction in the discovery cohort and the validation cohort, respectively. The number of patients with low-stage AKI and critical AKI was 2,132 (4.2%) and 605 (1.2%) in the discovery cohort. Among the discovery cohort patients with critical AKI, 511 (1.0%), 167 (0.3%) and 88 (0.2%) of the PO-AKI patients had at least two phases of AKI, post-AKI death, and dialysis within 90 days, respectively. The incidence of adverse outcomes gradually increased in the discovery cohort with 1,774 (4.5%) and 727 (1.8%) patients with low-stage AKI and critical AKI. Further, AKI in stage 2 or higher, post-AKI death and dialysis within 90 days were 644 (1.6%), 176 (0.4%) and 64 (0.2%) in the discovery cohort, respectively. Other characteristics of the above two cohorts were significantly different, as the validation cohort has consisted of older patients with higher male proportions (Table 1). Obstetrics and gynecology surgeries were relatively common in the discovery cohort, but orthopedic surgery appeared to occupy the largest part of the discovery cohort. Significant differences were also identified with respect to baseline experimental values and drug use.

TABLE 1

| | Discovery cohort | | Validation cohort | | |
|---|---|---|---|---|---|
| Variables | (N = 51,041) | | (N = 39,764) | | P |
| Demographics | | | | | |
| Age (years) | 56 | [44; 66] | 60 | [48; 70] | <0.001 |
| <40 | 9,206 | (18.0%) | 5,559 | (14.0%) | |
| ≥40 and <60 | 20,877 | (40.9%) | 13,492 | (33.9%) | |
| ≥60 and <80 | 19,684 | (38.6%) | 18,698 | (47.0%) | |
| ≥80 | 1,274 | (2.5%) | 2,015 | (5.1%) | |
| Sex | | | | | <0.001 |
| Female | 28,306 | (55.5%) | 18,706 | (47.0%) | |
| Male | 22,735 | (44.5%) | 21,058 | (53.0%) | |
| Body mass index (kg/m²) | 23.8 | [21.7; 26.0] | 24.1 | [21.9; 26.4] | <0.001 |
| <18.5 | 1,919 | (3.9%) | 854 | (3.8%) | |
| ≥18.5 and <30 | 45,098 | (91.5%) | 20,624 | (90.9%) | |
| ≥30 | 2,294 | (4.7%) | 1,218 | (5.4%) | |
| Preexisting comorbidities | | | | | |
| Heart disease | 1,629 | (3.2%) | 1,163 | (2.9%) | 0.022 |
| Hypertension | 9,824 | (19.2%) | 9,161 | (23.0%) | <0.001 |
| Diabetes mellitus | 3,956 | (7.8%) | 3,581 | (9.0%) | <0.001 |
| Surgery characteristics | | | | | |
| Departments | | | | | <0.001 |
| General surgery | 22,447 | (44.0%) | 14,733 | (37.1%) | |
| Neurosurgery | 5,063 | (9.9%) | 3,842 | (9.7%) | |
| Obstetrics and gynecology | 7,894 | (15.5%) | 908 | (2.3%) | |
| Orthopedics | 11,372 | (22.3%) | 16,823 | (42.3%) | |
| Urologic surgery | 4,265 | (8.4%) | 3,458 | (8.7%) | |
| Surgical duration (hours) | 2.2 | [1.5; 3.3] | 2.5 | [1.7; 3.6] | <0.001 |
| Expected surgical duration | 2.5 | [2.0; 3.0] | 3.0 | [2.0; 4.0] | <0.001 |
| (hours) | | | | | |
| Anesthesia type | | | | | <0.001 |
| General | 43,921 | (86.6%) | 30,570 | (76.9%) | |
| Non-general | 6,789 | (13.4%) | 9,194 | (23.1%) | |
| Emergency operation | 732 | (1.4%) | 1,965 | (4.9%) | <0.001 |
| Blood pressure (BP) before | | | | | |
| operation (mmHg) | | | | | |
| Systolic blood pressure | 124 | [113; 135] | 128 | [116; 142] | <0.001 |
| (SBP) | | | | | |
| Diastolic blood pressure | 77 | [70; 85] | 73 | [65; 81] | <0.001 |
| (DBP) | | | | | |
| Normotensive | 36,976 | (75.0%) | 22,934 | (57.7%) | |
| Hypertensive (SBP ≥140 or | 10,206 | (20.7%) | 11,472 | (28.9%) | |
| DBP ≥90) | | | | | |
| Hypotensive (SBP <90 or | 2,134 | (4.3%) | 5,358 | (13.5%) | |
| DBP <60) | | | | | |
| Medication usage | | | | | |
| RAAS blocker | 2,881 | (5.6%) | 2,915 | (7.3%) | <0.001 |
| Laboratory findings | | | | | |
| eGFR (mL/min/1.73 m²) | 82.1 | [71.4; 95.1] | 87.7 | [73.1; 99.7] | <0.001 |
| No CKD or CKD stage 1 or 2 | 46,971 | (92.0%) | 35,881 | (90.2%) | |
| (≥60) | | | | | |
| CKD stage 3A (≥45 and <60) | 3,226 | (6.3%) | 2,918 | (7.3%) | |
| CKD stage 3B (≥30 and <45) | 641 | (1.3%) | 703 | (1.8%) | |
| CKD stage 4 (≥15 and <30) | 203 | (0.4%) | 262 | (0.7%) | |
| Dipstick albuminuria (≥1+) | 4,682 | (9.3%) | 2,169 | (7.0%) | <0.001 |
| White blood cell count | 6,100 | [5,000; 7,500] | 6,500 | [5,400; 8,000] | <0.001 |
| (/mm²) | | | | | |
| Reference range | 43,262 | (84.8%) | 33,902 | (85.3%) | |
| (4000-10000) | | | | | |
| Leukopenia (<4000) | 3,934 | (7.7%) | 1,806 | (4.5%) | |
| Leukocytosis (≥10000) | 3,823 | (7.5%) | 4,033 | (10.1%) | |
| Hemoglobin (g/dL) | 13.2 | [12.1; 14.4] | 13.6 | [12.4; 14.8] | <0.001 |
| Anemia (<12 for female, <13 | 14,177 | (27.8%) | 9,212 | (23.2%) | <0.001 |
| for male) | | | | | |
| Platelet (×10³/μL) | 200 | [152; 256] | 239 | [199; 284] | <0.001 |
| Thrombocytopenia (<10) | 4,160 | (8.2%) | 493 | (1.2%) | <0.001 |
| Albumin (g/dL) | 4.2 | [3.9; 4.5] | 4.3 | [4.0; 4.5] | <0.001 |
| Hypoalbuminemia (<3.5) | 5,148 | (10.1%) | 2,679 | (6.8%) | <0.001 |
| Sodium (mEq/L) | 140 | [139; 142] | 141 | [139; 142] | <0.001 |
| Normonatremia (135~145) | 48,288 | (94.8%) | 31,156 | (92.8%) | |
| Hyponatremia (<135) | 1,291 | (2.5%) | 986 | (2.9%) | |
| Hypernatremia (>145) | 1,361 | (2.7%) | 1,418 | (4.2%) | |
| Potassium (mEq/L) | 4.2 | [4.0; 4.4] | 4.2 | [4.0; 4.5] | <0.001 |
| Normokaleima (3.5~5.5) | 49,711 | (97.6%) | 32,649 | (97.3%) | |
| Hypokalemia (<3.5) | 1,016 | (2.0%) | 711 | (2.1%) | |
| Hyperkalemia (>5.5) | 213 | (0.4%) | 200 | (0.6%) | |

CKD = chronic kidney disease

(2) Variable Selection

Table 2 shows patient characteristics according to the sequential results tested in the discovery cohort. In the cumulative logistic regression analysis, the surgical department, body mass index and blood pressure (BP) classification, anesthesia type, and hypernatremia were inappropriately satisfied in parallel regression estimation (Table 3), thus being excluded from construction of additional models. Further, the serum potassium level range and leukocytosis did not show a significant relationship with the sequential results in the multivariant proportional odds model (Table 4). Finally, heart disease, hypertension and leukopenia involved model coefficients relatively small as compared to others (Table 5), thus being excluded from the models. Other variables were included for final index construction and verification.

A total of 49,803 and 29,715 cases, respectively, in the discovery cohort and the validation cohort with complete information of the finally selected variables were used for further analysis in order to construct and verify simplified models (FIG. 2). The number of low-stage AKI patients was 2,062 (4.1%) and 1,109 (3.7%) in the discovery cohort and the validation cohort, respectively, with no missing values. Critical AKI appeared in perfect cases as follows: 563 persons (1.1%) in the discovery cohort and 445 persons (1.5%) in the validation cohort. Lastly, the model coefficients of the variables selected from the proportional odds model are shown in Table 6 below.

TABLE 2

| | No AKI | | Low-stage AKI | | Critical AKI | | |
|---|---|---|---|---|---|---|---|
| Variables | (N = 48,304) | | (N = 2,132) | | (N = 605) | | P |
| Demographics | | | | | | | |
| Age | 55 | [44; 66] | 63 | [53; 71] | 65 | [55; 73] | <0.001 |
| <40 | 9,016 | (18.7%) | 152 | (7.1%) | 38 | (6.3%) | |
| ≥40 and <60 | 19,990 | (41.4%) | 698 | (32.7%) | 189 | (31.2%) | |
| ≥60 and <80 | 18,194 | (37.7%) | 1,158 | (54.3%) | 332 | (54.9%) | |
| ≥80 | 1,104 | (2.3%) | 124 | (5.8%) | 46 | (7.6%) | |
| Sex | | | | | | | <0.001 |
| Female | 27,402 | (56.7%) | 706 | (33.1%) | 198 | (32.7%) | |
| Male | 20,902 | (43.3%) | 1,426 | (66.9%) | 407 | (67.3%) | |
| Body mass index | 23.8 | [21.6; 26.0] | 24.0 | [21.9; 26.3] | 23.7 | [21.3; 26.1] | 0.005 |
| (kg/m²) | | | | | | | |
| <18.5 | 1,802 | (3.9%) | 77 | (3.8%) | 40 | (7.1%) | |
| (underweight) | | | | | | | |
| ≥18.5 and <30 | 42,743 | (91.5%) | 1,854 | (90.4%) | 501 | (88.5%) | |
| (normal range) | | | | | | | |
| >30 (obesity) | 2,150 | (4.6%) | 119 | (5.8%) | 25 | (4.4%) | |
| Preexisting | | | | | | | |
| co-morbidities | | | | | | | |
| Heart disease | 1,445 | (3.0%) | 132 | (6.2%) | 52 | (8.6%) | <0.001 |
| Hypertension | 8,989 | (18.6%) | 653 | (30.6%) | 182 | (30.1%) | <0.001 |
| Diabetes mellitus | 3,494 | (7.2%) | 357 | (16.7%) | 105 | (17.4%) | <0.001 |
| Surgery | | | | | | | |
| characteristics | | | | | | | |
| Departments | | | | | | | <0.001 |
| General surgery | 20,962 | (43.4%) | 1,167 | (54.7%) | 318 | (52.6%) | |
| Neurosurgery | 4,920 | (10.2%) | 106 | (5.0%) | 37 | (6.1%) | |
| Obstetrics and | 7,802 | (16.2%) | 61 | (2.9%) | 31 | (5.1%) | |
| gynecology | | | | | | | |
| Orthopedics | 10,834 | (22.4%) | 476 | (22.3%) | 62 | (10.2%) | |
| Urologic surgery | 3,786 | (7.8%) | 322 | (15.1%) | 157 | (26.0%) | |
| Surgery duration | 2.2 | [1.4; 3.2] | 3.8 | [2.2; 5.8] | 3.9 | [2.3; 6.2] | <0.001 |
| (hours) | | | | | | | |
| Expected surgery | 2.5 | [2.0; 3.0] | 3.0 | [2.5; 5.0] | 3.0 | [2.5; 5.0] | <0.001 |
| duration (hours) | | | | | | | |
| Anesthesia type | | | | | | | <0.001 |
| General | 41,480 | (86.4%) | 1,878 | (88.6%) | 563 | (93.5%) | |
| Non-general | 6,508 | (13.6%) | 242 | (11.4%) | 39 | (6.5%) | |
| Emergency | 613 | (1.3%) | 74 | (3.5%) | 45 | (7.8%) | <0.001 |
| operation | | | | | | | |
| BP before | | | | | | | |
| operation | | | | | | | |
| SBP | 123 | [113; 135] | 126 | [114; 138] | 125 | [113; 137] | <0.001 |
| DBP | 77 | [70; 85] | 76 | [69; 84] | 75 | [68; 83] | <0.001 |
| Normotensive | 35,199 | (75.2%) | 1,389 | (69.4%) | 388 | (72.4%) | |
| Hypertensive (SBP ≥140 | 9,601 | (20.5%) | 493 | (24.6%) | 112 | (20.9%) | |
| or DBP ≥90) | | | | | | | |
| Hypotensive (SBP <90 | 1,978 | (4.2%) | 120 | (6.0%) | 36 | (6.7%) | |
| or DBP <60) | | | | | | | |
| Preoperative | 2,482 | (5.1%) | 294 | (13.8%) | 105 | (17.4%) | <0.001 |
| RAAS blocker | | | | | | | |
| Laboratory | | | | | | | |
| findings | | | | | | | |
| eGFR | 82.3 | [71.8; 95.1] | 76.9 | [61.9; 93.6] | 74.0 | [57.6; 92.9] | <0.001 |
| (mL/min/1.73 m²) | | | | | | | |
| No CKD or CKD | 44,900 | (93.0%) | 1,633 | (76.6%) | 438 | (72.4%) | |
| stage 1 or 2 (≥60) | | | | | | | |
| CKD stage 3A | 2,836 | (5.9%) | 311 | (14.6%) | 79 | (13.1%) | |
| (≥45 and <60) | | | | | | | |
| CKD stage 3B | 459 | (1.0%) | 132 | (6.2%) | 50 | (8.3%) | |
| (≥30 and <45) | | | | | | | |
| CKD stage 4 (≥15 | 109 | (0.2%) | 56 | (2.6%) | 38 | (6.3%) | |
| and <30) | | | | | | | |
| Presence of | 4,060 | (8.6%) | 427 | (20.5%) | 195 | (33.2%) | <0.001 |
| albuminuria | | | | | | | |
| (dipstick ≥1+) | | | | | | | |
| White blood cell | 6.1 | [5.0; 7.5] | 6.3 | [4.9; 7.9] | 6.6 | [4.9; 8.9] | <0.001 |
| count (1000/mm²) | | | | | | | |
| Reference range | 41,211 | (85.3%) | 1,627 | (76.3%) | 424 | (70.3%) | |
| (4000~10000) | | | | | | | |
| Leukopenia | 3,581 | (7.4%) | 278 | (13.0%) | 75 | (12.4%) | |
| (<4000) | | | | | | | |
| Leukocytosis | 3,493 | (7.2%) | 226 | (10.6%) | 104 | (17.2%) | |
| (≥10000) | | | | | | | |
| Hemoglobin (g/dL) | 13.3 | [12.1; 14.4] | 12.6 | [10.9; 14.0] | 12.1 | [10.4; 13.8] | <0.001 |
| Anemia (<12 for | 12,819 | (26.6%) | 1,010 | (47.4%) | 348 | (57.6%) | <0.001 |
| female, <13 for | | | | | | | |
| male) | | | | | | | |
| Platelet (10³/μL) | 200 | [152; 255] | 206 | [140; 294] | 207 | [135; 324] | <0.001 |
| Thrombocytopenia | 3,807 | (7.9%) | 263 | (12.6%) | 90 | (15.2%) | <0.001 |
| (<10) | | | | | | | |
| Albumin (g/dL) | 4.2 | [3.9; 4.5] | 3.9 | [3.3; 4.3] | 3.7 | [3.0; 4.2] | <0.001 |
| Hypoalbuminemia | 4,322 | (9.0%) | 591 | (27.7%) | 235 | (38.8%) | <0.001 |
| (<3.5) | | | | | | | |
| Sodium (mEq/L) | 140 | [139; 142] | 140 | [138; 142] | 140 | [137; 142] | <0.001 |
| Normonatremia | 45,900 | (95.2%) | 1,881 | (88.2%) | 507 | (83.8%) | |
| (135~145) | | | | | | | |
| Hyponatremia | 1,028 | (2.1%) | 190 | (8.9%) | 73 | (12.1%) | |
| (<135) | | | | | | | |
| Hypernatremia | 1,275 | (2.6%) | 61 | (2.9%) | 25 | (4.1%) | |
| (>145) | | | | | | | |
| Potassium (mEq/L) | 4.2 | [4.0; 4.4] | 4.2 | [4.0; 4.5] | 4.2 | [3.9; 4.5] | <0.001 |
| Normokaleima | 47,108 | (97.7%) | 2,041 | (95.7%) | 562 | (92.9%) | |
| (3.5~5.5) | | | | | | | |
| Hypokalemia | 920 | (1.9%) | 64 | (3.0%) | 32 | (5.3%) | |
| (<3.5) | | | | | | | |
| Hyperkalemia | 175 | (0.4%) | 27 | (1.3%) | 11 | (1.8%) | |
| (>5.5) | | | | | | | |

TABLE 3

| | (Low-stage AKI + Critical AKI) vs. (No AKI) | | | (Critical AKI) vs. (Low-stage AKI + No AKI) | | |
|---|---|---|---|---|---|---|
| | Coefficient | 95% CI | P | Coefficient | 95% CI | P |
| Age <40 | Reference | | | Reference | | |
| ≥40 and <60 | 0.745 | 0.588, 0.906 | <0.001 | 0.790 | 0.453, 1.154 | <0.001 |
| ≥60 and <80 | 1.357 | 1.207, 1.513 | <0.001 | 1.420 | 1.098, 1.773 | <0.001 |
| ≥80 | 1.989 | 1.772, 2.205 | <0.001 | 2.201 | 1.769, 2.640 | <0.001 |
| Male sex (vs. | 0.978 | 0.896, 1.060 | <0.001 | 0.951 | 0.782, 1.123 | <0.001 |
| female) | | | | | | |
| Departments | | | | | | |
| General | Reference | | | Reference | | |
| surgery | | | | | | |
| Neurosurgery | −0.891 | −1.069, −0.720 | <0.001 | −0.669 | −1.027, −0.341 | <0.001 |
| Obstetrics and | −1.793 | −2.012, −1.587 | <0.001 | −1.293 | −1.683, −0.941 | <0.001 |
| gynecology | | | | | | |
| Orthopedics | −0.355 | −0.457, −0.255 | <0.001 | −0.964 | −1.246, −0.698 | <0.001 |
| Urologic | 0.580 | 0.470, 0.688 | <0.001 | 0.978 | 0.782, 1.170 | <0.001 |
| surgery | | | | | | |
| BMI category | | | | | | |
| Underweight | 0.164 | −0.032, 0.351 | 0.093 | 0.639 | 0.299, 0.951 | <0.001 |
| (vs. reference | | | | | | |
| raneej | | | | | | |
| Obesity (vs. | 0.195 | 0.017, 0.365 | 0.028 | −0.019 | −0.449, 0.362 | 0.925 |
| reference | | | | | | |
| range) | | | | | | |
| Heart disease | 0.849 | 0.688, 1.005 | <0.001 | 1.069 | 0.769, 1.348 | <0.001 |
| (vs. none) | | | | | | |
| Hypertension | 0.652 | 0.567, 0.737 | <0.001 | 0.599 | 0.422, 0.772 | <0.001 |
| (vs. none) | | | | | | |
| Diabetes | 0.957 | 0.850, 1.062 | <0.001 | 0.932 | 0.714, 1.141 | <0.001 |
| mellitus (vs. | | | | | | |
| none) | | | | | | |
| Expected | 0.539 | 0.515, 0.563 | <0.001 | 0.458 | 0.417, 0.500 | <0.001 |
| surgery | | | | | | |
| duration | | | | | | |
| Non-general | −0.310 | −0.438, −0.185 | <0.001 | −0.810 | −1.151, −0.499 | <0.001 |
| anesthesia (vs. | | | | | | |
| general) | | | | | | |
| Emergency | 1.277 | 1.073, 1.474 | <0.001 | 1.803 | 1.477, 2.105 | <0.001 |
| operation (vs. | | | | | | |
| non- | | | | | | |
| BP category | | | | | | |
| Hypotensive | 0.222 | 0.126, 0.316 | <0.001 | 0.045 | −0.170, 0.253 | 0.675 |
| before surgery | | | | | | |
| (vs. | | | | | | |
| normotensive) | | | | | | |
| Hypertensive | 0.446 | 0.273, 0.613 | <0.001 | 0.481 | 0.120, 0.811 | 0.006 |
| before surgery | | | | | | |
| (vs. | | | | | | |
| normotensive) | | | | | | |
| Preoperative | 1.148 | 1.033, 1.260 | <0.001 | 1.282 | 1.064, 1.492 | <0.001 |
| RAAS | | | | | | |
| blockade use | | | | | | |
| (vs. no use) | | | | | | |
| eGFR ≥60 | Reference | | | Reference | | |
| ≥45 and <60 | 1.092 | 0.977, 1.206 | <0.001 | 0.981 | 0.732, 1.217 | <0.001 |
| ≥30 and <45 | 2.151 | 1.972, 2.327 | <0.001 | 2.196 | 1.881, 2.490 | <0.001 |
| ≥15 and <30 | 2.928 | 2.648, 3.207 | <0.001 | 3.197 | 2.819, 3.551 | <0.001 |
| Leukopenia | 0.683 | 0.564, 0.800 | <0.001 | 0.675 | 0.420, 0.916 | <0.001 |
| (vs. reference | | | | | | |
| range) | | | | | | |
| Leukocytosis | 0.641 | 0.518, 0.761 | <0.001 | 1.039 | 0.817, 1.251 | <0.001 |
| (vs. reference | | | | | | |
| range) | | | | | | |
| Anemia (vs. | 1.004 | 0.926, 1.081 | <0.001 | 1.280 | 1.118, 1.443 | <0.001 |
| none) | | | | | | |
| Hypoalbuminemia | 1.481 | 1.393, 1.568 | <0.001 | 1.772 | 1.605, 1.937 | <0.001 |
| (vs. none) | | | | | | |
| Hyponatremia | 1.593 | 1.450, 1.733 | <0.001 | 1.731 | 1.472, 1.976 | <0.001 |
| (vs. reference | | | | | | |
| range) | | | | | | |
| Hypernatremia | 0.260 | 0.030, 0.475 | 0.022 | 0.567 | 0.136, 0.950 | 0.006 |
| (vs. | | | | | | |
| reference | | | | | | |
| Hypokalemia | 0.636 | 0.416, 0.844 | <0.001 | 1.045 | 0.664, 1.390 | <0.001 |
| (vs. reference | | | | | | |
| range) | | | | | | |
| Hyperkalemia | 1.369 | 1.002, 1.710 | <0.001 | 1.561 | 0.888, 2.125 | <0.001 |
| (vs. reference | | | | | | |
| range) | | | | | | |
| Urine | 1.175 | 1.079, 1.270 | <0.001 | 1.605 | 1.429, 1.778 | <0.001 |
| albuminuria | | | | | | |
| (vs. none) | | | | | | |
| Thrombocytopenia | 0.564 | 0.446, 0.680 | <0.001 | 0.707 | 0.475, 0.928 | <0.001 |
| (vs. none) | | | | | | |

AKI = acute kidney injury,
CI = confidence interval,
BMI = body mass index,
BP = blood pressure,
RAAS = renin-angiotensin-aldosterone system,
eGFR = estimated glomerular filtration rate

Among the variables in Table 3 above, surgical departments such as General surgery, Neurosurgery, Obstetrics and gynecology, Orthopedics, Urologic surgery, bodyweight index (vs. reference range), obesity (vs. reference range), non-general anesthesia (vs. general), blood pressure (hypotensive before surgery (vs. normotensive), hypertensive before surgery (vs. normotensive)) and hypernatremia (vs. reference range) were not included in the construction of additional models due to significant differences in coefficients at each threshold. Further, parallel estimation could hardly be estimated with variables.

TABLE 4

| | Model | | |
|---|---|---|---|
| | coefficient | 95% CI | P |
| Age <40 | Reference | | |
| ≥40 and <60 | 0.473 | 0.302, 0.644 | <0.001 |
| ≥60 and <80 | 0.787 | 0.617, 0.956 | <0.001 |
| ≥80 | 1.120 | 0.875, 1.366 | <0.001 |
| Male sex (vs. female) | 0.714 | 0.624, 0.804 | <0.001 |
| Heart disease (vs. none) | 0.190 | 0.006, 0.374 | 0.042 |
| Hypertension (vs. none) | 0.176 | 0.069, 0.282 | 0.001 |
| Diabetes mellitus (vs. none) | 0.263 | 0.135, 0.391 | <0.001 |
| Expected surgery duration | 0.451 | 0.424, 0.478 | <0.001 |
| (hours) | | | |
| Emergency operation (vs. | 0.690 | 0.448, 0.931 | <0.001 |
| non-emergency) | | | |
| Preoperative RAAS blocker use | 0.473 | 0.338, 0.608 | <0.001 |
| (vs. none) | | | |
| eGFR ≥60 | | | |
| ≥45 and <60 | 0.663 | 0.532, 0.794 | <0.001 |
| ≥30 and <45 | 1.321 | 1.113, 1.528 | <0.001 |
| ≥15 and <30 | 1.921 | 1.607, 2.235 | <0.001 |
| Leukopenia (vs. reference range) | 0.395 | 0.254, 0.536 | <0.001 |
| Leukocytosis (vs. reference | 0.027 | −0.117, 0.172 | 0.712 |
| range) | | | |
| Anemia (vs. none) | 0.292 | 0.192, 0.392 | <0.001 |
| Hypoalbuminemia (vs. none) | 0.683 | 0.563, 0.802 | <0.001 |
| Hyponatremia (vs. reference | 0.307 | 0.162, 0.452 | <0.001 |
| range) | | | |
| Hypokalemia (vs. reference | 0.169 | −0.080, 0.418 | 0.184 |
| range) | | | |
| Hyperkalemia (vs. reference | 0.270 | −0.155, 0.696 | 0.212 |
| range) | | | |
| Urine albuminuria (vs. none) | 0.533 | 0.421, 0.646 | <0.001 |
| Thrombocytopenia (vs. none) | 0.041 | −0.090, 0.172 | 0.543 |

CI = confidence interval,
RAAS = renin angiotensin aldosterone system,
eGFR = estimated glomerular filtration rate

Among the variables in Table 4 above, leukocytosis (vs. reference range), hypokalemia (vs. reference range), hyperkalemia (vs. reference range), thrombocytopenia (vs. none) were not included in the construction of additional models due to significant differences in coefficients at each threshold.

TABLE 5

| | Model | | |
|---|---|---|---|
| | coefficient | 95% CI | P |
| Age (vs. <40) | | | |
| ≥40 and <60 | 0.502 | 0.332, 0.672 | <0.001 |
| ≥60 and <80 | 0.807 | 0.638, 0.976 | <0.001 |
| ≥80 | 1.136 | 0.892, 1.379 | <0.001 |
| Male sex (vs. female) | 0.705 | 0.616, 0.794 | <0.001 |
| Heart disease (vs. none) | 0.187 | 0.004, 0.370 | 0.040 |
| Hypertension (vs. none) | 0.171 | 0.065, 0.276 | <0.001 |
| Diabetes mellitus (vs. none) | 0.275 | 0.148, 0.401 | <0.001 |
| Expected surgical duration | 0.458 | 0.432, 0.484 | <0.001 |
| (continuous, hours) | | | |
| Emergency operation | 0.669 | 0.432, 0.906 | <0.001 |
| Preoperative RAAS blocker use (vs. | 0.453 | 0.319, 0.588 | <0.001 |
| none) | | | |
| eGFR (vs. ≥60 mL/min/1.73 m²) | | | |
| ≥45 and <60 | 0.680 | 0.551, 0.809 | <0.001 |
| ≥30 and <45 | 1.331 | 1.127, 1.536 | <0.001 |
| ≥15 and <30 | 2.021 | 1.714, 2.328 | <0.001 |
| Leukopenia (vs. none) | 0.204 | 0.097, 0.310 | <0.001 |
| Anemia (vs. none) | 0.305 | 0.206, 0.404 | <0.001 |
| Hypoalbuminemia (vs. none) | 0.667 | 0.550, 0.784 | <0.001 |
| Hyponatremia (vs. none) | 0.296 | 0.153, 0.438 | <0.001 |
| Albuminuria (vs. none) | 0.505 | 0.394, 0.617 | <0.001 |

CI = confidence interval,
RAAS = renin angiotensin aldosterone system,
eGFR = estimated glomerular filtration rate.

An additional simple model consists of age and additional 10 variables according to sizes of model coefficients. Further, among the variables in Table 5, heart disease (vs. none), hypertension (vs. none), and leukopenia (vs. none) were not included in the construction of additional models because of relatively small model coefficients.

TABLE 6

| | Model | | |
|---|---|---|---|
| | coefficients | 95% CI | P |
| Age (vs. <40) | | | |
| ≥40 and <60 | 0.522 | 0.353, 0.691 | <0.001 |
| ≥60 and <80 | 0.852 | 0.686, 1.019 | <0.001 |
| ≥80 | 1.203 | 0.962, 1.443 | <0.001 |
| Male sex (vs. female) | 0.705 | 0.616, 0.794 | <0.001 |
| Diabetes mellitus (vs. none) | 0.347 | 0.227, 0.467 | <0.001 |
| Expected surgical duration | 0.459 | 0.433, 0.484 | <0.001 |
| (continuous, hours) | | | |
| Emergency operation | 0.678 | 0.441, 0.915 | <0.001 |
| RAAS blocker use (vs. none) | 0.506 | 0.375, 0.638 | <0.001 |
| eGFR (vs. ≥60 mL/min/1.73 m²) | | | |
| ≥45 and <60 | 0.690 | 0.561, 0.818 | <0.001 |
| ≥30 and <45 | 1.345 | 1.141, 1.549 | <0.001 |
| ≥15 and <30 | 2.012 | 1.705, 2.319 | <0.001 |
| Anemia (vs. none) | 0.319 | 0.221, 0.418 | <0.001 |
| Hypoalbuminemia (vs. none) | 0.705 | 0.590, 0.820 | <0.001 |
| Hyponatremia (vs. none) | 0.298 | 0.156, 0.441 | <0.001 |
| Albuminuria (vs. none) | 0.510 | 0.399, 0.621 | <0.001 |

CI = confidence interval,
RAAS = renin angiotensin aldosterone system,
eGFR = estimated glomerular filtration rate
Model coefficients are multiplied by 11.0306, and rounded to the nearest integer to form the SPARK index.

(3) SPARK Index and Classification

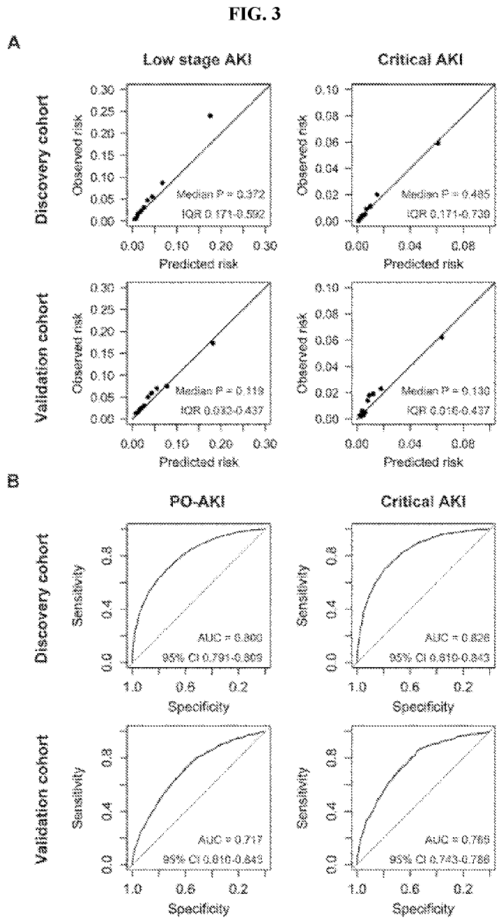

Selected variables were fitted to the sequential results with a proportional odds model. Although some under-estimation was found in the high probability range of the discovery cohort, the calibration plot showed an acceptable distribution of estimated and expected probabilities (FIG. 3). In 1000 random subsamples of fixed size (n=1,000), Hosmer-Lemeshow test provided median P values of 0.372 [90.9% of samples with interquartile range (IQR) of 0.171 to 0.592 and P≥0.05] and 0.485 [88.3% of samples with IQR of 0.171 to 0.739 and P≥0.05] in regard to low-stage AKI and critical MU results, respectively, in the discovery cohort. The median P values in the discovery cohort from the same test also demonstrated that the model suitability was significantly acceptable (P>0.05). However, as a small percentage of the sub-samples showed good model suitability, the calibration results were relatively favorable within the validation cohort. The median P value for low-stage AKI was 0.119 (67.8% of samples with IQR of 0.032 to 0.294 and P≥0.05], while the median P value for critical AKI was 0.130 (65.0% of samples with IQR of 0.016 to 0.437 and P≥0.05]. The c-stat was 0.798 and 0.715 in the discovery cohort and the validation cohort, respectively, which was within the acceptable range. After converting the model coefficients into an integer score, the final SPARK index showed acceptable discriminative ability in regard to all of the results such as PO-AKI [area under the discovery cohort curve (AUC) of 0.800 (95% CI 0.791-0.809), validation cohort AUC=0.717 (95% CI 0.705-0.730)] and critical AKI [discovery cohort AUC=0.826 (95% CI 0.810-0.843), validation cohort AUC=0.765 (95% CI 0.743-0.786)].

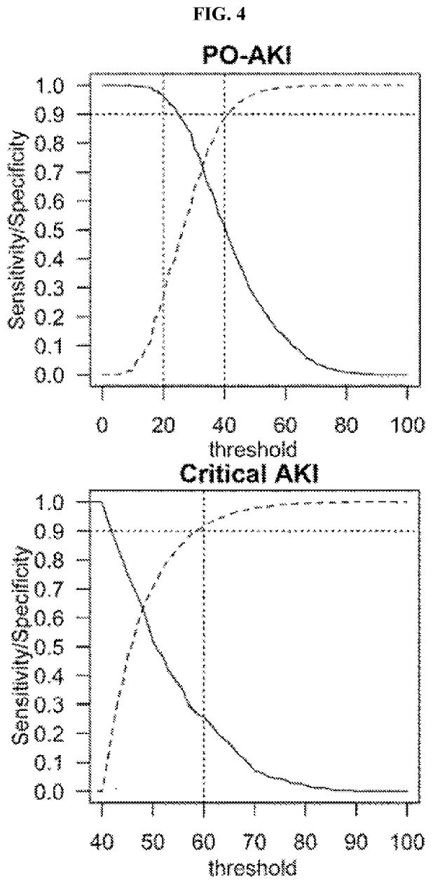

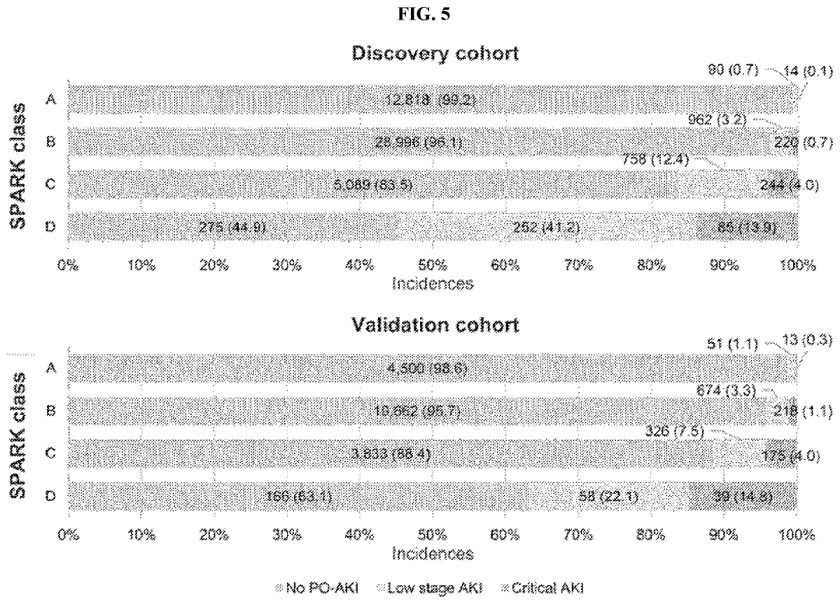

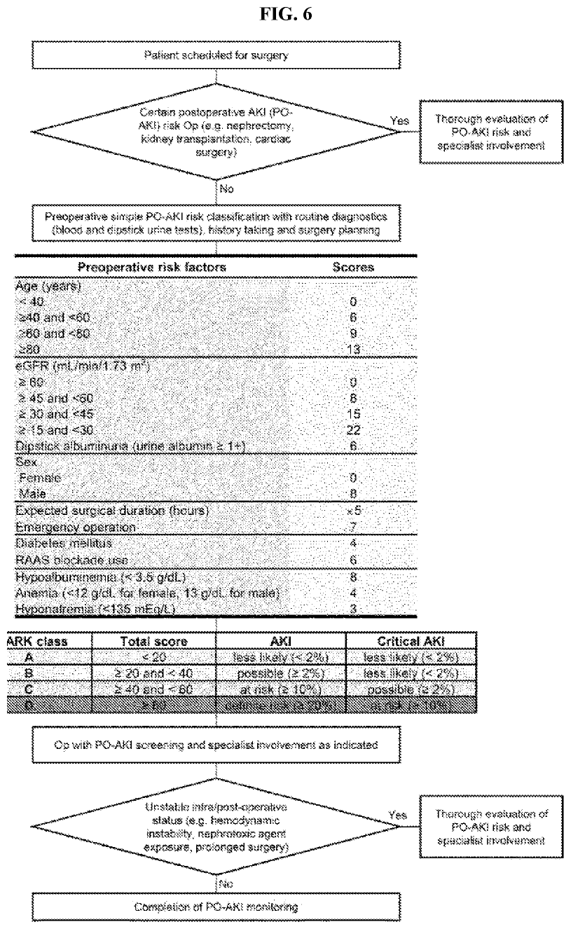

Subsequently, the sensitivity/specificity values were examined (FIG. 4), and the cutoff values of 20 and 40 were chosen as thresholds between SPARK grades A/B and B/C, respectively (Table 7). Further, a threshold of 60 was confirmed as the threshold between SPARK C and D grades. After completing the classification at a designated cutoff value, both the incidences of AKI and critical AKI demonstrated a grade-dependent increase in the discovery cohort and the verification cohort (FIG. 5). From the above mentioned results, the SPARK index and classification were constructed, while proposing a pre-operative AKI monitoring strategy (FIG. 6).

TABLE 7

| | | | Negative | Positive |
|---|---|---|---|---|
| | | | predictive | predictive |
| | Sensitivity | Specificity | value | value |
| PO-AKI | | | | |
| Discovery cohort | | | | |
| Cutoff = 20 | 96.0% | 27.2% | 99.2% | 6.8% |
| Cutoff = 40 | 51.0% | 88.6% | 97.0% | 20.0% |
| Validation cohort | | | | |
| Cutoff = 20 | 95.9% | 16.0% | 98.6% | 5.9% |
| Cutoff = 40 | 38.5% | 85.8% | 96.2% | 13.0% |
| Critical AKI (among | | | | |
| SPARK index ≥40) | | | | |
| Discovery cohort | | | | |
| Cutoff = 60 | 25.8% | 91.7% | 96.0% | 13.9% |
| Validation cohort | | | | |
| Cutoff = 60 | 18.2% | 94.9% | 96.0% | 14.8% |

PO-AKI = postoperative acute kidney injury,
AKI = acute kidney injury,
SPARK = simple postoperative acute kidney injury risk.

(4) Sensitivity Analysis

Sensitivity analysis was performed to investigate whether there was a significant bias due to exclusion criteria. The discriminative ability of the SPARK index in the attributed dataset including cases where missing values were present was acceptable in the discovery cohort [c-stat=0.802 (N=51,041)]. However, in the validation cohort with a high percentage of missing values, in which dipstick proteinuria variables were greatly missing, the discriminative ability was relatively reduced [c-stat=0.698 (N=39,764)]. When the actual surgical duration was included instead of the expected surgical duration, there was no significant decrease in discriminative ability [c-stat=0.810 in the discovery cohort (N=49,803) and c-stat=0.723 in the validation cohort (N=29,715)]. Then, after combining the discovery cohort and validation cohort, whether there are obvious differences therebetween over time was subjected to investigation. In the three periods of the present experiment, no significant difference or decrease in c-stat was observed: [C-stat=0.754 in 2004 to 2007 (N=18,560), c-stat=0.779 in 2008 to 2011 (N=34,016), and c-stat=0.768 in 2012 to 2015 (N=26,942)]. Lastly, in order to control a potential bias from a set of pre-operative sub-acute or chronic progressive kidney injury, patients with a pre-operative creatinine level obviously increased by 0.3 mg/dL or more or 1.5 times or more from the minimum value within 3 months prior to surgery were excluded regardless of duration. Further, even in the analysis, the discriminative ability of the SPARK index was maintained within the acceptable range [c-stat=0.792 in the discovery cohort (N=48,124) and c-stat=0.711 in the validation cohort (N=29,315)].

(5) Implementation of SPARK Index and Classification in Each Surgical Department

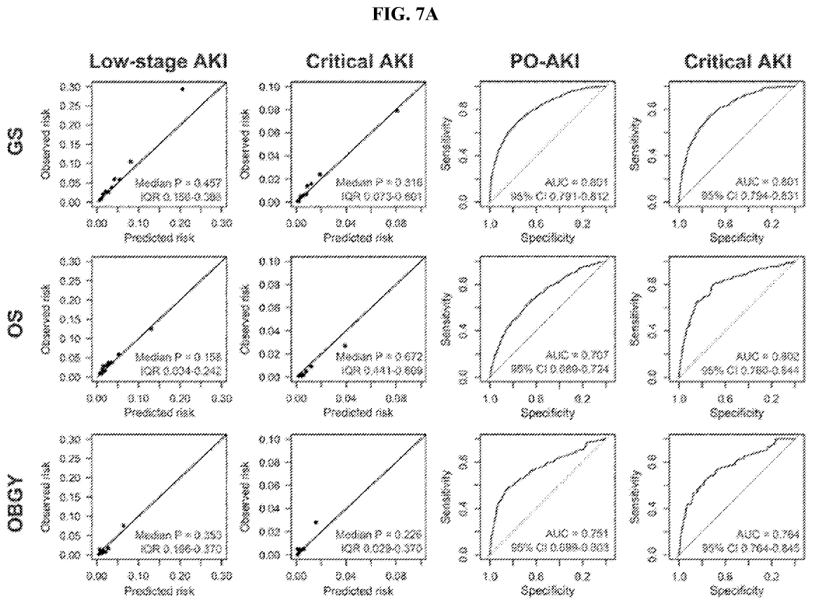

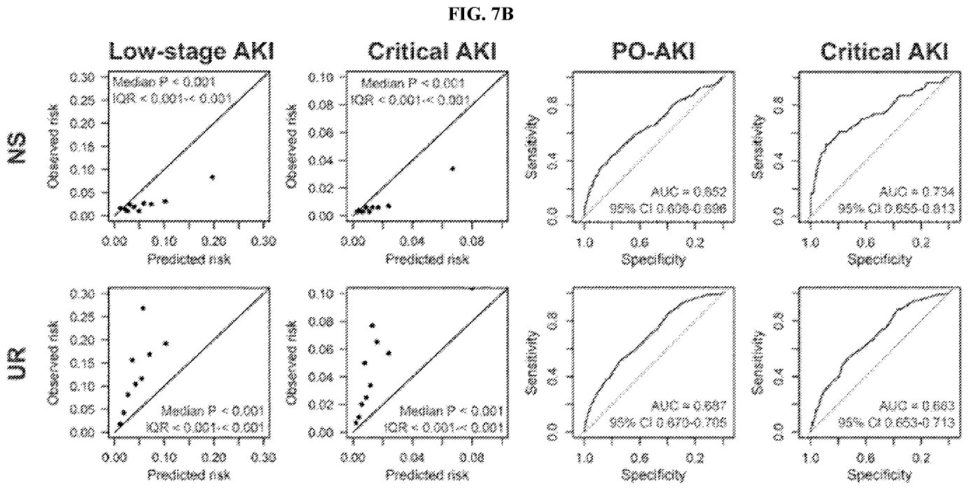

When merging the cases in the discovery and validation cohorts without missing values, certain differences related to clinical characteristics were present between surgery departments (Table 8). The results of the simplified proportional odds model and SPARK index implementation in each department are shown in FIGS. 7A and 7B. The compensation results indicated that all compensations were acceptable in general surgery and orthopedics, and were favorable in gynecology. However, in the model of the present invention, it was confirmed that the risk of adverse outcomes was under-estimated in the urology surgery, whereas was significantly over-estimated in the neurosurgery department. With regard to the discriminative ability, a similar tendency was demonstrated. More particularly, AUC values in the neurosurgery and urology departments were 0.7 or less, indicating relatively low discriminative ability. Nevertheless, when the SPARK classification was applied, a remarkable increase in incidence according to grades of adverse results was again observed (Table 9).

TABLE 8

| | | | | | Obstetrics | | | | | | |
|---|---|---|---|---|---|---|---|---|---|---|---|
| | General | | Orthopedic | | and | | | | Urologic | | |
| | surgery | | surgery | | gynecology | | Neurosurgery | | surgery | | |
| | (N = 31,810) | | (N = 24,873) | | (N = 8,351) | | (N = 7,280) | | (N = 7,204) | | P |
| Age (years) | 58 | [49; 68] | 59 | [44; 69] | 45 | [36; 52] | 56 | [45; 65] | 66 | [57; 72] | <0.001 |
| <40 | 3,023 | (9.5%) | 5,171 | (20.8%) | 2,827 | (33.9%) | 1,186 | (16.3%) | 692 | (9.6%) | |
| ≥40 and <60 | 13,949 | (43.9%) | 7,559 | (30.4%) | 4,332 | (51.9%) | 3,185 | (43.8%) | 1,473 | (20.4%) | |
| ≥60 and <80 | 13,869 | (43.6%) | 11,149 | (44.8%) | 1,133 | (13.6%) | 2,813 | (38.6%) | 4,780 | (66.4%) | |
| ≥80 | 969 | (3.0%) | 994 | (4.0%) | 59 | (0.7%) | 96 | (1.3%) | 259 | (3.6%) | |
| eGFR | 82.7 | [71.7; 95.2] | 85.7 | [72.6; 98.0] | 86.5 | [75.4; 100.8] | 85.5 | [72.8; 99.4] | 76.6 | [64.7; 88.2] | <0.001 |
| (mL/min/1.73 m²) | | | | | | | | | | | |
| ≥60 | 29,432 | (92.5%) | 22,516 | (90.5%) | 8,042 | (96.3%) | 6,740 | (92.6%) | 5,936 | (82.4%) | |
| ≥45 and <60 | 1,895 | (6.0%) | 1,830 | (7.4%) | 250 | (3.0%) | 440 | (6.0%) | 963 | (13.4%) | |
| ≥30 and <45 | 366 | (1.2%) | 401 | (1.6%) | 43 | (0.5%) | 74 | (1.0%) | 226 | (3.1%) | |
| ≥15 and <30 | 117 | (0.4%) | 126 | (0.5%) | 16 | (0.2%) | 26 | (0.4%) | 79 | (1.1%) | |
| Dipstick | 2,806 | (8.8%) | 1,459 | (5.9%) | 699 | (8.4%) | 402 | (5.5%) | 1,316 | (18.3%) | <0.001 |
| albuminuria | | | | | | | | | | | |
| Male sex | 16,900 | (53.1%) | 11,025 | (44.3%) | 0 | (0.0%) | 3,366 | (46.2%) | 6,455 | (89.6%) | <0.001 |
| Expected | 3.0 | [2.0; 3.5] | 2.5 | [2.0; 3.0] | 2.0 | [2.0; 3.0] | 4.0 | [3.0; 5.0] | 3.0 | [2.0; 4.0] | <0.001 |
| surgery duration | | | | | | | | | | | |
| (hours) | | | | | | | | | | | |
| Emergency | 444 | (1.4%) | 296 | (1.2%) | 164 | (2.0%) | 233 | (3.2%) | 58 | (0.8%) | <0.001 |
| department | | | | | | | | | | | |
| Diabetes | 2,705 | (8.5%) | 2,522 | (10.1%) | 255 | (3.1%) | 595 | (8.2%) | 597 | (8.3%) | <0.001 |
| mellitus | | | | | | | | | | | |
| RAAS blocker | 1,647 | (5.2%) | 1,756 | (7.1%) | 235 | (2.8%) | 712 | (9.8%) | 472 | (6.6%) | <0.001 |
| use | | | | | | | | | | | |
| Albumin (g/dL) | 4.2 | [3.9; 4.5] | 4.4 | [4.1; 4.6] | 4.3 | [4.1; 4.5] | 4.2 | [3.9; 4.5] | 4.4 | [4.2; 4.6] | <0.001 |
| Hypoalbuminemia | 3,126 | (9.8%) | 1,478 | (5.9%) | 554 | (6.6%) | 683 | (9.4%) | 197 | (2.7%) | |
| (<3.5) | | | | | | | | | | | |
| Hemoglobin | 13.3 | [12.0; 14.4] | 13.6 | [12.5; 14.9] | 12.7 | [11.6; 13.5] | 13.4 | [12.4; 14.5] | 14.3 | [13.2; 15.2] | <0.001 |
| (g/dL) | | | | | | | | | | | |
| Anemia (<12 | 9,425 | (29.6%) | 4,444 | (17.9%) | 2,555 | (30.6%) | 1,631 | (22.4%) | 1,280 | (17.8%) | |
| for female, <13 | | | | | | | | | | | |
| for male) | | | | | | | | | | | |
| Sodium | 141 | [139; 142] | 141 | [139; 142] | 140 | [139; 141] | 141 | [139; 142] | 141 | [139; 142] | <0.001 |
| (mEq/L) | | | | | | | | | | | |
| Hyponatremia | 933 | (2.9%) | 545 | (2.2%) | 102 | (1.2%) | 169 | (2.3%) | 111 | (1.5%) | |
| (<135) | | | | | | | | | | | |

eGFR = estimated glomerular filtration rate,
RAAS = rennin-angiotensin-aldosterone system

TABLE 9

| | Class A | | Class B | | Class C | | Class D | | P |
|---|---|---|---|---|---|---|---|---|---|
| General surgery | (N = 6,228) | | (N = 19,988) | | (N = 4,988) | | (N = 606) | | |
| Any AKI | 47 | (0.8%) | 785 | (3.9%) | 827 | (16.6%) | 347 | (57.3%) | <0.001 |
| Low-stage AKI | 41 | (0.7%) | 606 | (3.0%) | 610 | (12.2%) | 259 | (42.7%) | <0.001 |
| Critical AKI | 6 | (0.1%) | 179 | (0.9%) | 217 | (4.4%) | 88 | (14.5%) | <0.001 |
| Orthopedic surgery | (N = 6,119) | | (N = 16,776) | | (N = 1,908) | | (N = 70) | | |
| Any AKI | 78 | (1.3%) | 604 | (3.6%) | 219 | (11.5%) | 14 | (20.0%) | <0.001 |
| Low-stage AKI | 71 | (1.2%) | 539 | (3.2%) | 169 | (8.9%) | 8 | (11.4%) | <0.001 |
| Critical AKI | 7 | (0.1%) | 65 | (0.4%) | 50 | (2.6%) | 6 | (8.6%) | <0.001 |
| Obstetrics and gynecology | (N = 4,300) | | (N = 3,900) | | (N = 145) | | (N = 6) | | |
| Any AKI | 26 | (0.6%) | 68 | (1.7%) | 20 | (13.8%) | 1 | (16.7%) | <0.001 |
| Low-stage AKI | 17 | (0.4%) | 44 | (1.1%) | 13 | (9.0%) | 0 | (0.0%) | <0.001 |
| Critical AKI | 9 | (0.2%) | 24 | (0.6%) | 7 | (4.8%) | 1 | (16.7%) | <0.001 |
| Neurosurgery | (N = 287) | | (N = 5,133) | | (N = 1,781) | | (N = 79) | | |
| Any AKI | 7 | (2.4%) | 91 | (1.8%) | 76 | (4.3%) | 15 | (19.0%) | <0.001 |
| Low-stage AKI | 6 | (2.1%) | 71 | (1.4%) | 52 | (2.9%) | 6 | (7.6%) | <0.001 |
| Critical AKI | 1 | (0.3%) | 20 | (0.4%) | 24 | (1.3%) | 9 | (11.4%) | <0.001 |
| Urologic surgery | (N = 552) | | (N = 4,935) | | (N = 1,603) | | (N = 114) | | |
| Any AKI | 10 | (1.8%) | 526 | (10.7%) | 361 | (22.5%) | 57 | (50.0%) | <0.001 |
| Low-stage AKI | 6 | (1.1%) | 376 | (7.6%) | 240 | (15.0%) | 37 | (32.5%) | <0.001 |
| Critical AKI | 4 | (0.7%) | 150 | (3.0%) | 121 | (7.5%) | 20 | (17.5%) | <0.001 |

AKI = acute kidney injury

1: A system for predicting a risk of acute kidney injury after non-cardiac surgery, the system comprising:
a variable selection unit configured to select factors associated with an occurrence of acute kidney injury after non-cardiac surgery, which include clinical data before non-cardiac surgery of patients subjected to non-cardiac surgery as variables, and preset an index set for each variable;
a classification reference point (“classification cutoff value”) setting unit configured to calculate sensitivity and specificity for a sum of combinations of indexes for each index set, and set cutoff values according to the sensitivity and specificity; and
a prediction unit for the risk of acute kidney injury after non-cardiac surgery, which is configured to calculate a sum of indexes determined according to the index set preset for each variable selected by the variable selection unit in regard to a patient subjected to non-cardiac surgery in need of a prediction, and then, predict the risk of acute kidney injury after non-cardiac surgery of the non-cardiac surgery patient in need of the prediction, on the basis of cutoff values set in the classification cutoff value setting unit,
wherein the factors associated with the occurrence of acute kidney injury after non-cardiac surgery include at least one selected from the group consisting of age, estimated glomerular filtration rate (eGFR), dipstick albuminuria, sex, expected surgical duration, emergency operation, diabetes mellitus, use of renin-aldosterone-angiotensin-system blocker (use of RAAS blocker), hypoalbuminemia, anemia and hyponatremia.

2: The system according to claim 1, wherein the index set is preset according to a selection category for each variable except for the expected surgical duration, at least one index selected from 0, 3, 4, 6 to 9, 13, 15 and 22 is preset for each selection category, and a sum of the indexes for each selection category of the variable is 0 to 81.

3: The system according to claim 1, wherein the index preset for the expected surgical duration among the variables is set to 5 times the expected surgical duration (hours).

4: The system according to claim 1, wherein, among the variables, the age is divided into selection categories of less than 40 years old, 40 or more and less than 60 years old, 60 or more and less than 80 years old, and not less than 80 years old, and
wherein the estimated glomerular filtration rate (eGFR) is divided into selection categories of 60 mL/min/1.73 m² or more, 45 mL/min/1.73 m² or more and less than 60 mL/min/1.73 m², 30 mL/min/1.73 m² or more and less than 45 mL/min/1.73 m², 15 mL/min/1.73 m² or more and less than 30 mL/min/1.73 m².

5: The system according to claim 1, wherein the factors associated with the occurrence of acute kidney injury after non-cardiac surgery include age, estimated glomerular filtration rate (eGFR), dipstick albuminuria, sex, expected surgical duration, emergency operation, diabetes mellitus, use of renin-aldosterone-angiotensin-system blocker, hypoalbuminemia, anemia and hyponatremia.

6: The system according to claim 1, wherein the prediction of the risk of acute kidney injury after non-cardiac surgery is classified into total four (4) grades including A, B, C and D, based on a sum of indexes determined according to the index set preset for each variable selected in the variable selection unit in regard to the non-cardiac surgery patient in need of the prediction.

7: The system according to claim 6, wherein the grade A is classified when the sum of the indexes is less than 20, the grade B is classified when the sum of the indexes is 20 or more and less than 40, the grade C is classified when the sum of the indexes is 40 or more and less than 60, and the grade D is classified when the sum of the indexes is 60 or more.

8: The system according to claim 7, wherein the grade A involves less than 2% probability of both acute kidney injury and severe acute kidney injury after non-cardiac surgery of patients subjected to non-cardiac surgery (“non-cardiac surgery patients”) in need of the prediction;
the grade B involves 2% or more probability of acute kidney injury and less than 2% probability of serious acute kidney injury after non-cardiac surgery of the non-cardiac surgery patients in need of the prediction;
the grade C involves 10% or more probability of acute kidney injury and 2% or more probability of serious acute kidney injury after non-cardiac surgery of the non-cardiac surgery patients in need of the prediction; and
the grade D involves 20% or more probability of acute kidney injury and 10% or more probability of serious acute kidney injury after non-cardiac surgery of the non-cardiac surgery patients in need of the prediction.

9: A method for predicting a risk of acute kidney injury after non-cardiac surgery, the method comprising:
a first step of selecting factors associated with an occurrence of acute kidney injury after non-cardiac surgery, which consist of clinical data before non-cardiac surgery of patients subjected to non-cardiac surgery as variables, and then, presetting an index set for each variable;
a second step of calculating sensitivity and specificity to a sum of combinations of indexes for each index set, and setting cutoff values for classification according to the sensitivity and specificity; and
a third step of calculating a sum of indexes determined according to the index set preset for each variable selected in the first step in regard to the non-cardiac surgery patient in need of a prediction, and then, predicting a risk of acute kidney injury after non-cardiac surgery of the non-cardiac surgery patient on the basis of the cutoff values set in the second step,
wherein the factors associated with the occurrence of acute kidney injury after non-cardiac surgery include at least one selected from the group consisting of age, estimated glomerular filtration rate (eGFR), dipstick albuminuria, sex, expected surgical duration, emergency operation, diabetes mellitus, use of renin-aldosterone-angiotensin-system blocker (use of RAAS blocker), hypoalbuminemia, anemia and hyponatremia.

10: The method according to claim 9, wherein the index set is preset according to a selection category for each variable except for the expected surgical duration, at least one index selected from 0, 3, 4, 6 to 9, 13, 15 and 22 is preset for each selection category, and a sum of the indexes for each selection category of the variable is 0 to 81.

11: The method according to claim 9, wherein the index preset for the expected surgical duration among the variables is set to 5 times the expected surgical duration (hours).

12: The method according to claim 9, wherein, among the variables, the age is divided into selection categories of less than 40 years old, 40 or more and less than 60 years old, 60 or more and less than 80 years old, and not less than 80 years old, and
wherein the estimated glomerular filtration rate (eGFR) is divided into selection categories of 60 mL/min/1.73 m² or more, 45 mL/min/1.73 m² or more and less than 60 mL/min/1.73 m², 30 mL/min/1.73 m² or more and less than 45 mL/min/1.73 m², 15 mL/min/1.73 m² or more and less than 30 mL/min/1.73 m².

13: The method according to claim 9, wherein the factors associated with the occurrence of acute kidney injury after non-cardiac surgery include age, estimated glomerular filtration rate (eGFR), dipstick albuminuria, sex, expected surgical duration, emergency operation, diabetes mellitus, use of renin-aldosterone-angiotensin-system blocker (use of RAAS blocker), hypoalbuminemia, anemia and hyponatremia.

14: The method according to claim 9, wherein the prediction of the risk of acute kidney injury after non-cardiac surgery is classified into total four (4) grades including A, B, C and D, based on a sum of indexes determined according to the index set preset for each variable selected in the variable selection unit in regard to the non-cardiac surgery patient in need of the prediction.

15: The method according to claim 14, wherein the grade A is classified when the sum of the indexes is less than 20, the grade B is classified when the sum of the indexes is 20 or more and less than 40, the grade C is classified when the sum of the indexes is 40 or more and less than 60, and the grade D is classified when the sum of the indexes is 60 or more.

16: The method according to claim 15, wherein the grade A involves less than 2% probability of both acute kidney injury and severe acute kidney injury after non-cardiac surgery of patients subjected to non-cardiac surgery (“non-cardiac surgery patients”) in need of the prediction;
the grade B involves 2% or more probability of acute kidney injury and less than 2% probability of serious acute kidney injury after non-cardiac surgery of the non-cardiac surgery patients in need of the prediction;
the grade C involves 10% or more probability of acute kidney injury and 2% or more probability of serious acute kidney injury after non-cardiac surgery of the non-cardiac surgery patients in need of the prediction; and
the grade D involves 20% or more probability of acute kidney injury and 10% or more probability of serious acute kidney injury after non-cardiac surgery of the non-cardiac surgery patients in need of the prediction.
